(19) United States
(12) Patent Application Publication
(10) Pub. No.: US 2010/0168578 A1
(43) Pub. Date: Jul. 1, 2010

(54) System and Method for Combined ECG-Echo for Cardiac Diagnosis

(75) Inventors: Arthur Garson, JR., Charlottesville, VA (US); William F. Walker, Earlysville, VA (US); John A. Hossack, Charlottesville, VA (US); Travis N. Blalock, Charlottesville, VA (US)

Correspondence Address: UNIVERSITY OF VIRGINIA PATENT FOUNDATION, 250 WEST MAIN STREET, SUITE 300, CHARLOTTESVILLE, VA, 22902, US

(73) Assignee: UNIVERSITY OF VIRGINIA PATENT FOUNDATION, Charlottesville, VA (US)

(21) Appl. No.: 12/664,146

(22) PCT Filed: Jun. 12, 2008
(86) PCT No.: PCT/US08/66711

§ 371 Date: Dec. 11, 2009

Related U.S. Application Data
(60) Provisional application No. 60/934,228, filed on Jun. 12, 2007.

(51) Int. Cl. A61B 8/14; A61B 5/0402

(52) U.S. Cl. 600/443; 600/509

(57) ABSTRACT

A system and related method for obtaining volumetric cardiac data of a subject. The data is generated by forming a plurality of focused ultrasound images corresponding to a series of ranges, generating myocardial boundary data for each of the plurality of ultrasound images, calculating the area of the region defined by said myocardial boundary data for each of the plurality of ultrasound images, multiplying the area for each of the plurality of ultrasound images by a slice depth corresponding to said ultrasound image to obtain the slice volume of each slice, and summing the slice volumes to obtain a total volume. In an alternative embodiment the system and related method combine an automated volumetric ultrasound system for finding chamber volumes and myocardial thicknesses, with a diagnostic electrocardiogram system to enable simultaneous diagnosis of mechanical and electrical cardiac problems.

### RELATED APPLICATIONS

The present invention claims priority from U.S. Provisional Application Ser. No. 60/934,228, filed Jun. 12, 2007, entitled “System and Method for Combined ECG-Echo for Cardiac Diagnosis,” which is hereby incorporated by reference herein in its entirety.

### GOVERNMENT SUPPORT

Work described herein was supported by Federal Grant Nos. EB002349 and EB001826, awarded by the NIH. The Government has certain rights to the invention.

### BACKGROUND

The primary function of the heart is as a contractile pump that transports blood to the lungs and throughout the body. While the heart's main function is as a mechanical pump, this pump is driven by an intricate electrical system. Cardiac dysfunction can result from mechanical dysfunction (i.e. poor contractility), electrical dysfunction (i.e. poor conductivity), or complex coupled electrical and mechanical problems. Electrical dysfunction is generally diagnosed clinically using a 12-lead electrocardiogram (ECG). Diagnostic ECG has the advantage of being relatively inexpensive, easy to use, and portable.

Electrical activity in the heart is commonly measured using the electrocardiogram (ECG). In its simplest common configuration the ECG acquires electrical activity using three electrodes to form the so-called 3 lead ECG. While the 3 lead ECG provides information about the rhythm of the heart, and can detect gross abnormalities such as left ventricular fibrillation, it is of no value for general cardiac diagnosis. The significant limitations of the 3 lead ECG are overcome somewhat by the more sophisticated 12 lead and 7 lead ECG configurations. Because these configurations are dramatically more useful, they are known as diagnostic ECG configurations. Diagnostic ECG is best suited for diagnosing electrical problems with the heart, but as will be described below, it is widely used to grossly estimate other quantities.

Mechanical parameters are also critical for accurate diagnosis. It is important to have accurate measurements of the size of the heart's chambers, the thickness of the walls and septa between the chambers as well as the change in chamber size during the cardiac cycle which is an indicator of the heart's function. It is well known by cardiologists that both static and dynamic measures of these dimensions provide critical information on conditions including hypertension, cardiomyopathy, and congenital heart disease. Only relatively expensive and complex imaging tools, such as echocardiography, are currently able to reliably measure critical dimensions.

The challenges associated with direct measurement of cardiac dimensions, volumes, and contractility via medical imaging has led clinicians to apply the diagnostic ECG as an indirect measure of cardiac dimensions. Specific dimensions of interest including chamber volumes, wall thickness, and septal thickness can only be indirectly assessed by diagnostic ECG with overall poor predictive value for these measurements. Historically the diagnostic ECG has achieved wide spread clinical application because of its ease of use and relatively low cost, even though it fails to accurately reflect chamber size and wall thickness. The diagnostic ECG does provide important information on heart rhythm, possible ischemia, infarction and other electrophysiological abnormalities. While the echocardiogram (echo) does directly reflect chamber size and wall thickness, traditional echo systems are costly, bulky, require a highly trained technician, and yield results with high dependence upon operator skill and experience. Further, by using separate ultrasound imaging and diagnostic ECG systems, it may be difficult for the clinician to correlate mechanical behavior and electrical behavior, undermining diagnosis.

The ECG was first developed in 1913 and utilized limb leads to measure the electrophysiological function of the heart. This simple configuration remains almost unaltered in the common 3-lead ECG systems used for patient monitoring and used to provide basic timing information in existing ultrasound imaging systems. While the primary use of the ECG in current ultrasound systems is to simply indicate when images were acquired relative to systole and diastole, in some ultrasound systems these ECGs are used to control the timing of acquisition and thereby synchronize acquisition with the cardiac cycle. These 3-lead ECGs and closely related 5-lead ECGs provide little or no diagnostic value. 3-Lead ECGs can be radically improved through the addition of unipolar chest leads, to construct the well-known diagnostic 12-lead ECG configuration. The diagnostic ECG is essential for determining heart rate and rhythm as well as electrophysiologic data such as the QT interval. It also displays conduction disturbance (such as bundle branch block) and is indicative of ischemia and infarction. Measurement of the size and shape of the P wave and QRS complexes has been correlated with atrial and ventricular dimension and wall thickness, but the diagnostic accuracy of diagnostic ECG for chamber size and hypertrophy is unacceptably poor. For example, a recent study found that only 6% of the variation in LV mass could be accounted for on the diagnostic ECG (Correlation of Electrocardiogram with Echocardiographic left ventricular mass in adult Nigerians with systemic hypertension, West African Journal of Medicine Vol. 22(3) 2003: 246-249, of which is hereby incorporated by reference).

A major problem with the diagnostic ECG is its unreliability in determining normality or abnormality of chamber size and wall thickness. The diagnostic ECG has been surpassed in this area by the echo, and the diagnostic ECG should not be measured or interpreted for such measurements. Notably, diagnostic ECG systems are relatively low in cost and only limited training is required to obtain high quality recordings.

Ultrasound imaging of the heart increased dramatically in the 1970's with the advent of the first phased array imaging systems. Modern 2D echocardiography systems can acquire high resolution images in vivo at frame rates in excess of 50 Hz, enabling visualization of moving structures and dynamic changes in chamber geometries, wall motion, and well/septal thicknesses. In the past five years, as 2D transducer arrays have become commercially viable, real-time 3D ultrasound imaging has entered mainstream clinical practice. The transition to 3D imaging is typically accompanied by a reduced frame-rate, somewhat reduced spatial resolution, and an increase in system cost. These negatives are offset however as 3D imaging provides the opportunity to determine chamber volumes, wall thicknesses, septal thicknesses, and other key parameters unambiguously. These applications are increasingly being supported by sophisticated software tools that automate much of the analysis and provide numerical and/or graphical output of these key parameters. Although ultrasound shows increasing potential in cardiac applications, high cost and overwhelming system complexity has greatly limited the scope of application.

Modern cardiac ultrasound imaging systems almost universally incorporate a simple 3-lead ECG system. The 3-lead ECG is deeply integrated in the system so that echo and ECG data can be displayed simultaneously and synchronously. One such system is described in U.S. Pat. No. 6,312,381 “Medical Diagnostic Ultrasound System and Method” which is herein incorporated by reference. While the incorporation of a 3-lead ECG indicates when images were acquired relative to systole and diastole, this simple ECG configuration is inadequate for diagnosing electrical dysfunction or identifying interacting electrical and mechanical problems.

### BRIEF SUMMARY

An aspect of various embodiments of the present invention comprises a device, system, method and computer program method that provides, among other things, the low cost, compact size, ease of use, and simple output format of diagnostic ECG and its electrophysiologic information combined with automated quantitative volume, dimensional, and contractile information available from echo. The present system and related methods described herein eliminate inaccurate analysis of the diagnostic ECG wave forms for chamber size and hypertrophy. The device, system, method and computer program method also alleviates the need for a highly trained technician. Accordingly, these advantages allow the proposed device and related methods to replace nearly all standard electrocardiographs.

An aspect of an embodiment of the current invention provides a combined ECG-echo system and related method that encompasses the advantage of each technology to optimize cardiac diagnosis and monitoring. In an embodiment the proposed invention incorporates a conventional multi-lead diagnostic ECG where the V4 lead is replaced by a combined ECG lead and low profile ultrasound transducer. In addition, an embodiment includes an automated ultrasound data acquisition and processing unit to extract dimensional, volumetric, and contractility/strain information from acquired ultrasound data. It should be appreciated that the ultrasound transducer may be placed at a different typical electrode location or even at a location distinct from normal location. Further, it will be realized that a two dimensional array is generally preferred as it enables the acquisition of true three dimensional (volumetric) information.

In an embodiment, an automated image processing system extracts dimensional, volumetric, and contractility/strain information that is acquired in synchrony with and displayed in graphical format along with traditional diagnostic ECG plots.

Another aspect of an embodiment presents images or maps showing regional contractility of the heart, as determined via ultrasound data. Such data may be superimposed with the estimated ECG vector.

The preferred system also has the ability to simultaneously acquire diagnostic ECG data with diagnostic ultrasound data. Existing methods require two separate patient studies and the clinician must correlate the results, attempting to mentally account for intervening changes in patient condition. The system and method described herein not only speeds the acquisition of diagnostic data, by combining both studies, but also eliminates the potential difficulties of correlating ECG and Echo data acquired at different times. The system also enables diagnosis of more subtle conditions which can only be observed by examining ECG and Echo data from the heart cycle or portion thereof.

Some exemplary and non-limiting novel aspects associated with various embodiments of the present invention include, but are not limited thereto, the following:
Echo combined with diagnostic ECG (note existing echo systems do not have the multiple lead display to enable electrophysiologic diagnosis).
A display combining graphs of chamber volumes, wall thickness and/or septal thickness with diagnostic ECG wave forms. (See FIG. 5 showing one embodiment.)
An ultrasound transducer with integrated ECG electrode.
An automated system for quantifying chamber size, septal and wall thicknesses throughout the cardiac cycle using ultrasound image data.
Automated diagnostic system which incorporates both ECG and ultrasound data as its inputs.
A twelve lead ECG system with integrated echo transducer.
An ultrasound transducer with low profile so as to allow placement for continuous monitoring. The transducer and associated hardware includes numerous aspects that improve the efficiency of the ultrasound data collection and image formation that enable the construction of the low-profile transducer. In one embodiment, the transducer may incorporate A/D conversion circuitry that uses direct inphase and quadrature (IQ) sampling of the received echo signal to reduce the amount of data samples that are required, thereby greatly reducing the complexity of the converter and reducing the heat generated by the circuit. Beamforming circuitry may also be integrated into the transducer and may generate image data points from combinations of rotated versions of the direct sampled IQ samples. Apodization weighting factors may be combined with the required phase rotations. The beamformers may operate to generate c-mode images from samples obtained over an echo time window limited to echoes associated with a desired c-mode image depth. Thus, forming c-mode images also limits the number of samples required to be obtained, further simplifying the data sampling and storage requirements. Still further, multiple image points may be generated in a serial fashion from the data set acquired from a single transmit firing event by re-processing the acquired data set with appropriate phase rotations (to simulate delays).
The transducer may use an oil-based, as opposed to water-based, couplant so as to be non-drying and thus avoiding the inevitable, and rapid, dryout problem with conventional couplants. Alternatively, the system may use a gel based couplant, like that commonly used on ECG leads, to maintain good coupling with very slow drying. Alternatively the system may use a gel-based couplant connected to a fluid reservoir so that liquid lost to the environment is replaced by liquid from the reservoir.
A transducer designed with low mass and minimal cabling to maximize positional stability. Such a transducer is particularly useful for continuous cardiac monitoring and for measurement during stress tests.
An interactive system that guides the placement of the echo transducer. Such interactive system may continuously output measures of image quality, such as mean brightness or image contrast, either audibly or visibly, so as to guide the user in placing the transducer in a more effective location.
An ultrasound transducer with integrated display or other feedback mechanism to guide the user in ultrasound transducer placement.
An assembly incorporating an ultrasound transducer with multiple ECG electrodes.
The system described above utilizing multiple ultrasound transducers in sequence or simultaneously.
The described system utilizing an ultrasound transducer intentionally designed to be larger than the available acoustic window. An associated electronic system which selects an appropriately sized and positioned subarray to access the proper active array position to image the desired internal features.
A non-drying gel for coupling the transducer to the patient. In an embodiment the gel is oil based, rather than the traditional water based formulation, to reduce drying. Alternatively, the system may use a gel based couplant, like that commonly used on ECG leads, to maintain good coupling with very slow drying. Alternatively the system may use a gel-based couplant connected to a fluid reservoir so that liquid lost to the environment is replaced by liquid from the reservoir.
An ultrasound transducer with associated adhesive structure for placement and retention on the patient.

### SUMMARY OF THE DRAWINGS

FIG. 1: Block diagram of the combined Ultrasound/ECG device. Note that this embodiment includes the standard 10 electrode configuration used in a 12 lead diagnostic ECG.

FIG. 2(A): Schematic diagram depicting electrode placement for R, L, N, and F leads of a standard 12 lead ECG.

FIG. 2(B): Schematic diagram depicting electrode placement for R, L, N, and F leads of a standard 12 lead ECG.

FIG. 2(C): Schematic diagram depicting electrode placement for v1, v2, v3, v4, v5, and v6 leads of a standard 12 lead ECG. Note that in one embodiment of the present invention the v4 electrode is a combined ECG electrode and ultrasound transducer assembly.

FIG. 3(A): Schematic diagram of an exterior view of a 2D array (optimized to be hand-held).

FIG. 3(B): Schematic diagram of an exterior view of a low-profile 2D array designed for long-term placement on the patient, without manual intervention.

FIG. 4: Diagram showing automated aperture selection.

FIG. 5: One embodiment of an output report prepared by the proposed system.

FIG. 6: A flow chart illustrating the data flow in an embodiment.

### DETAILED DESCRIPTION

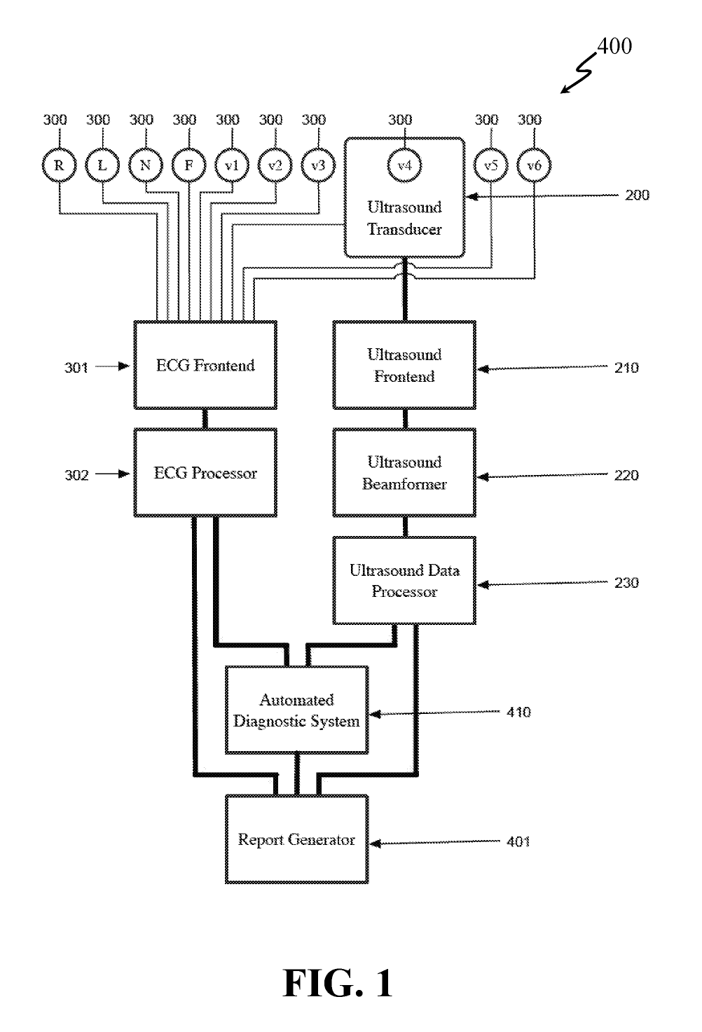

Referring generally to FIGS. 1-6, an exemplary approach of the present invention includes a system 400 for obtaining volumetric cardiac data 404 of a subject 100, comprising an ultrasound transducer 200, an ultrasound beamformer 220 connected to said transducer adapted to create focused ultrasound data 403 corresponding to a volume within the subject, means for generating myocardial boundary data 408 from said focused ultrasound data 403, described below, means for generating volumetric data 404 using said myocardial boundary data, described below, and a real-time display means, storage means, or both, for outputting said volumetric data 404. In an embodiment, the ultrasound transducer 200 comprises a two-dimensional transducer array capable of generating fully three-dimensional image data.

The ultrasound beamformer 220 may incorporate a transmit beamformer 222. The transmit beamformer 222 generates transmit ultrasound signals 252 which are passed to the transducer 221 where they produce a transmitted ultrasound waveform. Note that the transmit beamformer 222 may incorporate focusing using either time delays or phase delays, or may use a simple plane wave transmission scheme to minimize hardware complexity. The ultrasound beamformer device 220 also incorporates a receive beamformer 224 that processes received ultrasound echo data 254 to form a focused ultrasound data set 403. A variety of known beamforming methods may be applied by the receive beamformer 224. One preferred approach is the DSIQ beamforming algorithm, described in more detail below This method is extremely efficient in terms of computational operations and hardware and thus may enable low-cost and compact imaging and monitoring applications. The DSIQ beamforming algorithm specifically forms a c-scan image at a given range from the transducer. Those of ordinary skill in the art will appreciate that a set of such images formed at different ranges effectively forms a volumetric data set.

In an embodiment, the system further comprises an electrocardiograph (ECG) module for receiving ECG signals from the subject. The ECG can be, but does not have to be, a standard 7- or 12-lead ECG. The ECG waveforms 406 are also displayed, stored, or both along with the volumetric cardiac data 404.

In an embodiment, the means for generating said myocardial boundary data 408 comprises first applying an envelope detector 232 to the focused ultrasound data 403 to yield envelope detected ultrasound data 256. A next step in this embodiment entails either selecting a slice from within the volume of data, or selecting a single image plane, such as might be formed by DSIQ beamforming, and then applying active contours to the envelope detected ultrasound data 256 to define the myocardial regions. The active contour method will be applied in an edge detection block 234 which may be implemented in hardware, software, or some combination of the two. It should be readily apparent that one may repeat the above process on a series of slices to obtain a volumetric data set defining the tissue boundaries. Alternatively the system may detect boundaries natively on the 3D data set. In an embodiment, the SRAD algorithm is applied to the envelope detected ultrasound data 256 to reduce image speckle before applying active contours.

In one embodiment the means for generating volumetric data 404 comprises determining the area of the region defined by a single slice of said myocardial boundary data 408, determining a slice thickness for each of the plurality of ultrasound images 403, multiplying said area by said slice thickness to obtain a slice volume for each of the set of ultrasound data 403, and summing said slice volumes to obtain a total volume. The slice thickness may be, but does not have to be, one half of the distance between the image and the previous image, plus one half of the distance between the image and the next image. In a system employing DSIQ beamforming, which preferentially forms c-scan images at different ranges, the thickness of a slice may be estimated using slice ranges as an equivalent of the distances described above. As with other portions of the system, the volume estimator 236 may be implemented in software, hardware, or some combination of the two.

An embodiment includes an automated sub-system for determining the thicknesses of various anatomical features of the heart, including the septum, ventricular wall, and atrial wall. This thickness estimating subsystem 238 accepts the myocardial boundary data 408 and processes that data to yield specific thickness measurements 258. In one embodiment the thickness estimator 238 accepts inner and outer myocardial boundary locations 408 and determines the distance between the inner and outer myocardial boundaries at some user selected location to determine the myocardial thickness. Alternatively the thickness estimator 238 determines the minimum distance between the inner myocardial boundaries of the left and right ventricles to determine the septal thickness. Alternatively the septal thickness might be determined at a specific distance form the heart's apex or at some percentage of the length of the heart. Many other variations of thickness measurement will be readily apparent to one of ordinary skill in the art. Because the estimated myocardial boundaries may be noisy and rough, it may be advantageous to smooth these boundaries prior to estimating thicknesses.

In many applications it may be advantageous to determine how effectively the heart is contracting. Possibly the most thorough way to assess this is to compute the local strains throughout the myocardium. Such an operation can be performed by applying a strain estimator 240 to the focused ultrasound data produced by the receive beamformer. Computation of the strain will of course require processing of at least two acquisitions from the same tissue region, thus the strain estimator or other system components incorporates a memory to hold focused echo data from multiple acquisitions. The strain field 260 can be computed by the strain estimator 240 by taking the spatial gradient of estimated displacements or by directly computing the strain using higher order methods. Exemplary strategies for strain estimation are discussed below.

While using strain to quantify cardiac contractility is very thorough, it has the limitation of being computationally costly. In some cases a much cruder measure of contractility can be estimated by quantifying changes in the thickness of the myocardium. Such estimation may be performed by processing the edge data 408 to determine the local and/or average distances between the inner and outer myocardial boundaries. The performance of this method may be improved by constraining results to meet the required conservation of myocardial volume using the method described in “Guiding automated left ventricular chamber segmentation in cardiac imaging using the concept of conserved myocardial volume” (Comput Med Imaging Graph. 2008 Apr. 7; 32 (4):321-330) which is hereby incorporated by reference.

An embodiment includes an automated diagnostic system 410 for generating diagnostic data 407 from various combinations of the volumetric cardiac data 404, the ECG waveforms 406, the envelope data 256, the edge definitions (boundaries) 408, the various thickness data 258, the strain field 260, and other available information sources. This automated diagnostic system 410 does not have to be physically distinct from the ultrasound data processor 230; the different computational steps may be completed on the same hardware, for example by a general purpose computer, but do not have to be. In an embodiment, the ECG waveforms 406 and focused ultrasound data 403 are fed to a low-power application-specific signal processor which is adapted to generate the volumetric cardiac data 404 from the focused ultrasound data 403 and also to generate the diagnostic information 407 from the volumetric cardiac data 404 and ECG waveforms 406.

An embodiment may further include a transducer 200 that integrates an ultrasound transducer and an ECG electrode. One aspect of an embodiment is that the transducer comprises a two-dimensional ultrasound transducer array 206 capable of generating true three-dimensional image data. Another aspect of an embodiment is that the transducer be a low-profile device 202 suitable to continuous use rather than acute diagnosis.

A low profile transducer without an integrated ECG electrode represents yet another embodiment of the invention. The transducer preferably has a cable that leaves the transducer housing parallel to the active transducer face, rather than leaving it perpendicular to the active face, as is known in prior art systems. A preferred embodiment for the low-profile transducer uses a flat cable assembly, rather than the prior art cylindrical cables, so that the cable may be laid flat against the subject. Such a design makes the transducer less obtrusive and makes it less likely that forces on the cable would act to move the transducer from its preferred location. The transducer cable is desired to be as flexible as possible to minimize transducer motion.

An embodiment of the present invention may further include a method for obtaining volumetric cardiac data 404 of a subject 100, comprising the steps of forming a plurality of focused ultrasound data 403 corresponding to a series of ranges (possibly using the DSIQ beamforming algorithm), generating myocardial boundary data 408 for each of the plurality of ultrasound data 403 from specific ranges, calculating the area of the region defined by said myocardial boundary data 408 for each of the plurality of ultrasound data 403, multiplying the area calculated for each of the plurality of ultrasound data 403 by a slice depth corresponding to said ultrasound data 403 to obtain the slice volume of each slice, summing the slice volumes to obtain a total volume, using said volumetric data 404 to generate diagnostic information 407, and outputting said diagnostic information 407 to either a real-time display means, a storage device, or both.

It should be noted that the calculations are independent of the data gathering steps, so the volumetric data 404 can be calculated contemporaneously with each ultrasound data 403 acquisition, or alternatively they can be calculated as a batch for an entire sequence of ultrasound data sets 403. Either method is encompassed within the description of preferred embodiments.

It will should be appreciated that the image and signal processing and analysis methods applied within the disclosed system may have some difficulty in operating in a fully automated manner. Such difficulties arise because of poor ultrasound image quality including shadowing, reverberation, and other non-ideal image features. To enable robust operation the disclosed system may accept user input to guide boundary detection or other operations. Such user input may be provided at the onset of data acquisition and then may be optionally applied periodically over time to obtain continuous robust results without continued interaction from the user.

As an alternative to accepting user input, the system attempts to fit a model to the data from a pre-acquired library of expected results. Such model fitting allows the system to robustly fit areas with poor image quality or other limitations. Models may be effectively represented using principal component analysis so that the system does not fit any exact model from the library, but is rather fitting the aspects of “typical” data sets.

In an embodiment, the method also includes gathering ECG waveforms 406 from the subject 100 and incorporating said ECG waveforms 406 into the diagnostic information 407.

In an embodiment, an ECG is connected to the subject, and is a standard 10 electrode configuration used in a 12 lead diagnostic ECG. In one embodiment, a unique feature is the combination of an ultrasound transducer with electrode v4 200. In addition to comprising a coupled ECG and ultrasound imaging system, the ultrasound system includes a greater degree of image processing than is seen in conventional systems. Another differentiating feature is the combined Ultrasound/ECG diagnostic system 400 and the combined report generator 401.

In an embodiment, a conventional 2D ultrasound transducer array 201, optimized to be hand-held, is used. In another embodiment, and a low-profile 2D transducer array 202 designed for long-term placement on the patient without manual intervention is used. For the low-profile transducer array 202 the mounting tab 203 may be covered with adhesive, attached to a strap placed around the patient's chest, or taped in place.

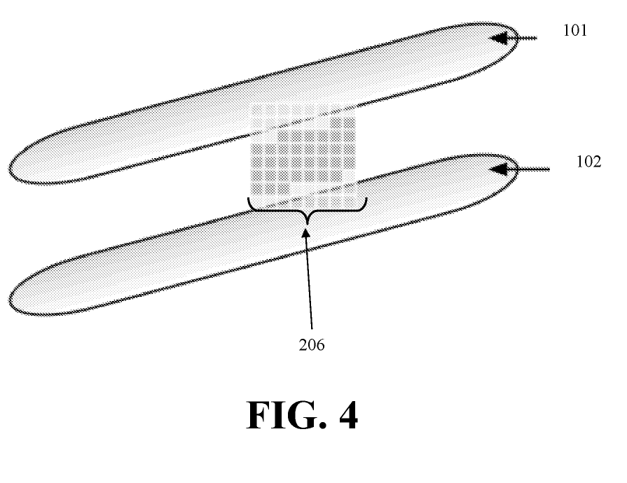

Referring to FIG. 4, in an embodiment, a transducer array 206 is placed roughly between a first rib 101 and a second rib 102. The system automatically detects the presence of a rib 101 in front of the array 206 and utilizes only those elements that have a clear acoustic view of the heart. Blocked elements are disabled, preferably by ultrasound front end 210, and are not used for imaging. In one embodiment the ultrasound front end 210 causes the transducer 200 to emit an ultrasound pulse from each element and disables any element that receives an echo with amplitude above a set threshold arriving before a set range in tissue. Such an approach detects the strong local echoes from the first rib 101 and second rib 102 for example.

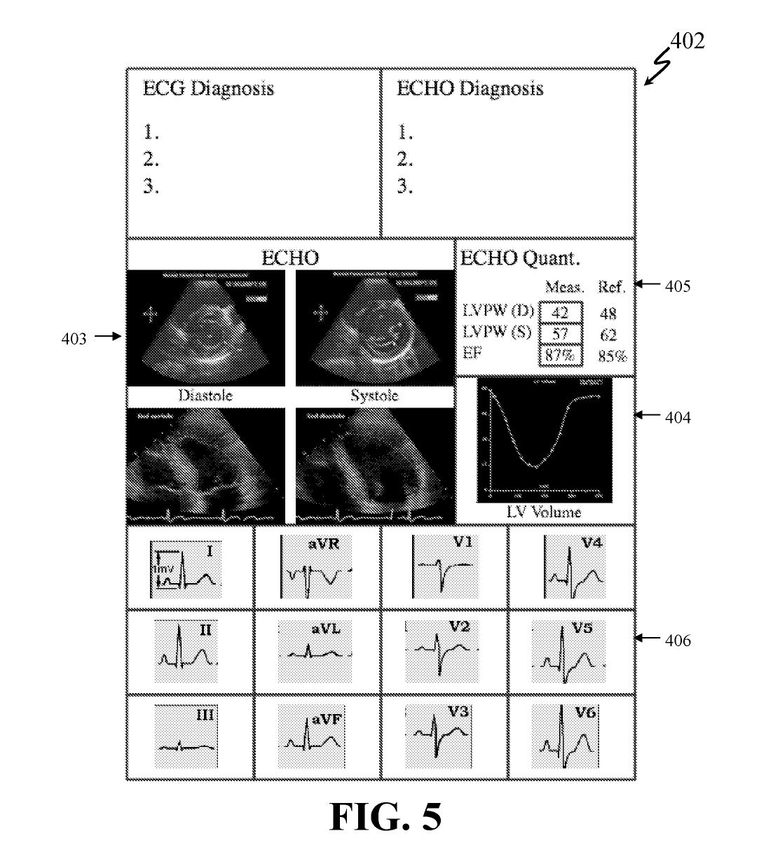

Referring to FIG. 5, in an embodiment, a report generator 401 generates a report 402 which summarizes the diagnostic conclusions from both echo and ECG. Sample ultrasound images 403 are shown to substantiate the automated analysis and justify the conclusions drawn, a graph showing volumetric information 404 is shown, numerical measures of cardiac performance 405 are depicted with normal ranges, and standard ECG waveforms 406 are shown. Alternate embodiments may graph septal thickness, atrial volumes, right ventricular volume, and other parameters. One knowledgeable in the art will appreciate that many other combinations of ECG and echo derived data might be displayed.

An aspect of an embodiment of the present invention is a system and related method that, among other things, integrates the electrophysiological measurement functions of an ECG with volume and automated geometric measurement functions performed via ultrasound. This yields, among other things, a more powerful tool for the diagnosis, screening, and monitoring of cardiac conditions. A goal of an embodiment of the present invention is to yield a system that is low in cost and easy to use, to maximize its clinical utility. A further goal of an embodiment of the present invention is a system that is highly portable and therefore appropriate for a broad range of applications.

In one embodiment, the present invention is a system and related method designed to be used for cardiac diagnosis and screening. The system and related method is preferably applied to the patient in much the same manner as a diagnostic 12 lead ECG system, however one or more of the ECG electrodes 300 is replaced by a combined ultrasound transducer and ECG lead assembly 200. The system simultaneously acquires multi-lead ECG measurements and multidimensional ultrasound data. In an embodiment the captured ultrasound data is volumetric in nature, enabling accurate measurement of parameters including chamber volumes, wall thicknesses, and septal thicknesses. The latter is particularly important as it can act as a predictor of the risk of sudden cardiac death. Chamber volumes, in particular dynamic measurements of chamber volumes, are indicative of cardiac function. In an embodiment, the invention utilizes automated image processing and segmentation to make the aforementioned measurements with little or no input from the user.

Preferably the ECG measurement data and ultrasound data includes timing information to permit the synchronization of the parameter measurements with the ECG waveform displays. Timing information may be managed by a synchronization module that is part of the automated diagnostic system 410. The synchronization module preferably communicates with the ECG processor 302 and the ultrasound data processor 230.

In one embodiment, the synchronization module periodically adds timing information to both the ECG and ultrasound data records as the data is being acquired and stored.

In another embodiment, synchronization information is obtained by grouping ECG lead waveform data and ultrasound volumetric data together. Preferably the synchronization module associates the ultrasound and ECG data sets with each other via data records, such as by placing the acquired ultrasound and ECG data samples into the same data record or into linked data records. In one embodiment, the synchronization module identifies characteristics of a cardiac contraction event and responsively associates the ECG waveform samples corresponding to the event with the ultrasound volumetric acquisitions corresponding to the event. The event may be identified using either the ECG data, the ultrasound data, or a combination of the two data. In this way, corresponding ECG and ultrasound data for individual events may be analyzed.

Referring to FIG. 1, a block diagram of the envisioned system 400 is shown. This embodiment incorporates many of the desired features of the invention. ECG electrode v4 300 is combined with an ultrasound transducer 200 so that the ECG technician can place the transducer on the patient or subject (not shown) as if they were simply placing a standard ECG electrode. Some additional training may be needed to ensure that the tech places the ultrasound transducer reliably and repeatably, however the required skill level is much lower than that required for a typical ultrasound tech as image quality is not the primary concern. The ultrasound data processing block 230 of the system could also include a feedback mechanism to indicate to the technician the quality of their transducer placement. The system 400 further comprises the ultrasound front-end 210, beamformer 220, the ECG front end 301, and ECG processor blocks 302, which are well known to those knowledgeable in the respective arts. An automated diagnostic system 410 for generating diagnostic data (not shown) and a report generator 401 for outputting, among other things, said diagnostic data to a real-time display means, storage means, or both (not shown) are also provided.

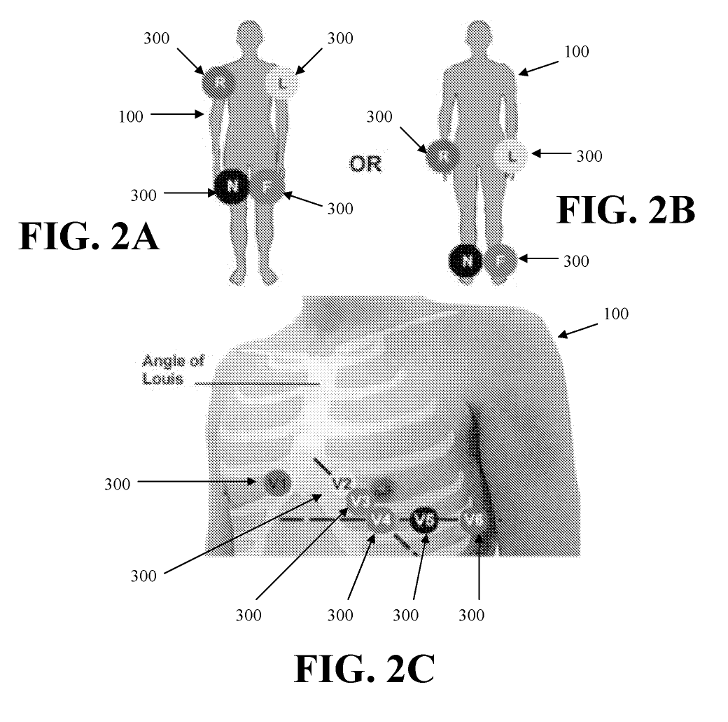

Turning to FIGS. 2(A)-2(C), a diagram showing the placement of the 10 electrodes needed for a 12 lead ECG configuration is shown. FIG. 2(A) shows a placement of the R, L, N, and F electrodes 300 on the subject 100. FIG. 2(B) shows another placement of the R, L, N, and F electrodes 300 on the subject 100. FIG. 2(C) shows a placement of the v1-v6 electrodes 300 on the subject 100. Note that in one embodiment the v4 electrode 300 is replaced by a combined ECG electrode/ultrasound transducer assembly 200.

While the high level functionality of the described invention has clear value, the detailed implementation of such a system is not trivial and requires various systems, devices, methods and a number of technologies. While a variety of commercially available systems are capable of forming volumetric images at high frame rates using two-dimensional sensor arrays, existing systems (see, for example, the Mark I from Volumetrics, the Vivid 7 from GE, and the SONOS 7500 and i22 from Philips) are bulky, expensive, and require significant user training and experience. An aspect associated with an embodiment of the present invention is that it would be, but not limited thereto, more easily implemented using the Sonic Window system described in a series of U.S. Patents and U.S. and PCT Patent Applications and of which are hereby incorporated by reference herein in their entirety:

1. “Efficient Architecture for 3D and Planar Ultrasonic Imaging—Synthetic Axial Acquisition and Method Thereof,” J. A. Hossack, T. N. Blalock, and W. F. Walker, PCT Application No. PCT/US/2005/036077, filed Oct. 5, 2005, and corresponding U.S. patent application Ser. No. 11/245,266,filed Oct. 5, 2005.

2. “Efficient Ultrasound System for Two-Dimensional C-Scan Imaging and Related Method Thereof,” J. A. Hossack, W. F. Walker, and T. N. Blalock, PCT Application No. PCT/US/2004/001002, filed Jan. 15, 2004, and corresponding U.S. patent application Ser. No. 10/542,242, filed Jul. 14, 2005.

3. “Ultrasonic Imaging Beam-former Apparatus and Method,” T. N. Blalock, W. F. Walker, and J. A. Hossack, PCT Application No. PCT/US2004/000887, filed Jan. 14, 2004, and corresponding U.S. application Ser. No. 11/160,915, filed Jul. 14, 2005.

4. “Ultrasonic Transducer Drive,” T. N. Blalock, W. F. Walker, and J. A. Hossack, PCT Application No. PCT/US2004/000888, filed Jan. 14, 2004, and corresponding U.S. application Ser. No. 11/160,914, filed Jul. 14, 2005.

5. “Intuitive Ultrasonic Imaging System and Related Method Thereof,” W. F. Walker, T. N. Blalock, and J. A. Hossack, PCT Application No. PCT/US2003/006607, filed Mar. 6, 2003, and U.S. patent application Ser. No. 10/506,722, filed Sep. 7, 2004.

These publications describe the use of a 2-dimensional transducer and associated hardware, which includes numerous aspects that improve the efficiency of the ultrasound data collection and image formation thereby enabling the construction of the low-profile transducer for use in the various embodiments described herein.

In one embodiment, the transducer may incorporate analog-to-digital (A/D) conversion circuitry that uses direct inphase and quadrature (IQ) sampling of the received echo signal. The A/D converters may include sample-and-hold circuits that are timed to capture samples at a known interval (preferably equal to one quarter wavelength of the ultrasound center frequency). In one embodiment, there are preferably two sample and hold circuits per transducer element, but four may be used to generate two IQ pairs, or other arrangements may be used to generate the direct sampled IQ data. The sample and hold voltages are then converted to digital values via an A/D circuit. Preferably, one A/D converter provides digital conversion for multiple sample and hold circuits to improve efficiency. The digital samples may then be used to generate a complex sample containing magnitude and phase information of the echo sample. The direct sampling to obtain the IQ data provides an efficient way to perform envelope detection that vastly reduces the amount of data samples that are required. That is, the direct sampling IQ technique is used to generate an abbreviated data record as compared to a full-rate A/D converter as is used in prior art ultrasound devices. The abbreviated data record preferably contains one or two IQ sample pairs, or up to as many as only sixteen or thirty-two pairs. As such, the complexity and rate of the A/D converter circuitry is reduced, as is the heat generated by the circuit.

Beamforming circuitry generates image data points from combinations of rotated versions of the direct sampled IQ samples. Apodization weighting factors may be combined with the required phase rotations. The beamformers may also operate to generate c-mode images from samples obtained over an echo time window limited to echoes associated with a desired c-mode image depth.

In another embodiment, an abbreviated data record may contain sequential data rather than IQ Pairs. The abbreviated record having sequential data may be generated according to the short time period that is relevant for the particular c-mode slice being generated. In one preferred embodiment, the abbreviated data records are no longer than thirty-two digital samples in length. The samples may be formed by a plurality of sample and hold circuits that are then processed by an A/D converter. Preferably, the A/D converter operates in a serial manner on the outputs from a plurality of sample and hold circuits. The sample and hold voltages are selectively connected to the A/D converter for conversion. The ratio of sample and hold circuits to A/D converters may be varied based on the speed of the A/D converter, the desired length of the data record, the sampling interval, etc. In this way, a slower speed A/D converter may be used, thereby reducing the amount of circuitry and amount of heat generated by the transducer assembly. In one embodiment, eight sample and hold circuits and a single A/D converter are proved for each receive transducer element, or receive channel. Thus, forming c-mode images also limits the number of samples required to be obtained for each transmit firing event, further simplifying the data sampling and storage requirements as compared to prior art full-rate sampling techniques.

Still further, one or more entire image planes may be generated from data captured from a single transmit firing. Further, multiple image points may be generated in a serial fashion from the data set acquired from a single transmit firing event by re-processing the acquired data set with appropriate delays or phase rotations in the case of direct sampled IQ data sets. In addition, samples from successive transmit firing events may be interleaved and/or concatenated prior to processing by the beamformer.

The Sonic Window provides a low cost and easy to use front-end for the acquisition of volumetric data. Once acquired, significant image processing is required to extract the key parameters needed for diagnosis (chamber volumes, wall thicknesses, etc.). It should be appreciated that numerous possible methods for achieving these tasks are possible, however we describe one strategy in detail, as an example. In one embodiment the system would perform the following tasks:

1. Emit a single unfocused transmit beam insonifying the entire field of view.

2. Simultaneously acquire echo data from the entire 2D transducer array. Digitize this echo data and store it in a memory for processing.

3. Form a focused ultrasound image at a given range using the DSIQ beamforming algorithm (Ranganathan, K., M. K. Santy, T. N. Blalock, J. A. Hossack, and W. F. Walker, “Direct Sampled IQ Beamforming for Compact and Very Low Cost Ultrasound Beamforming,” IEEE Trans. Ultrason. Ferroelec. Freq. Contr., vol. 51, no. 9, pp. 1082-94, 2004.), the entirety of which is hereby incorporated by reference herein.

4. Apply the SRAD algorithm to reduce the appearance of image speckle (Y. Yu and S. T. Acton, “Speckle Reducing Anisotropic Diffusion,” IEEE Transactions on Image Processing, vol. 11, pp. 1260-70, 2002.), the entirety of which is hereby incorporated by reference herein.

5. Apply active contours to detect myocardial boundaries (X. Fang, Y. Yongjian, S. T. Acton, and J. A. Hossack, “Detection of myocardial boundaries from ultrasound imagery using active contours,” presented at 2003 IEEE Ultrasonics Symposium. Honolulu, HI, USA. 5-8 Oct. 2003.), the entirety of which is hereby incorporated by reference herein.

6. Count pixels inside the active contour (or between contours) to quantify the area of interest.

7. Multiply the area determined in step 6 by the slice thickness to determine the partial volume of the region of interest for this slice.

8. Repeat steps 3-7 over a series of ranges to determine the volumes for each slice.

9. Sum the per slice volumes to determine the total volumes within the image volume.

The steps outlined above represent only one of many possible approaches executed by the system 400.

The performance of the methods outlined above may be further enhanced by the application of spatial compounding (G. E. Trahey, S. W. Smith, and O. T. v. Ramm, “Speckle pattern correlation with lateral aperture translation: experimental results and implications for spatial compounding,” IEEE Transations on Ultrasonics, Ferroelectrics, and Frequency Control, vol. UFFC-33:3, pp. 257-264, 1986, the entirety of which is incorporated herein by reference.). In the above implementation, spatial compounding is employed in receive only mode. In this mode a series of images are formed with different spatial origins for the receive aperture so that unique speckle patterns is acquired by each. These unique speckle patterns are then averaged so that a speckle reduced image results. This processing may be incorporated in step 3 and all further processing would remain the same. (It is possible that SRAD may be unnecessary in this case and may be eliminated to save computational costs.)

In cases where simple wall thicknesses are required the processing steps outlined above may be readily simplified so that the distances (mean or minimum) between active contours are measured. This further simplifies computation.

In cases where the user desires to measure the strength of contraction, the system may employ image or volume processing methods to calculate local displacements and then take a numerical gradient of the measured displacement field to determine strain. Displacements can be computed using cross-correlation, the sum-absolute-differences method, normalized correlation, or any of a number of other pattern matching techniques. Alternate methods such as optical flow may also be employed to estimate the displacement field. Displacement fields may also be computing using the MUltidimensional Spline-based Estimator (MUSE) described in U.S. Patent Application “Method, System, and Computer Apparatus for Registration of Multi-Dimensional Data Sets,” which is herein incorporated by reference. The MUSE method can also be employed to compute strain directly from the image data.

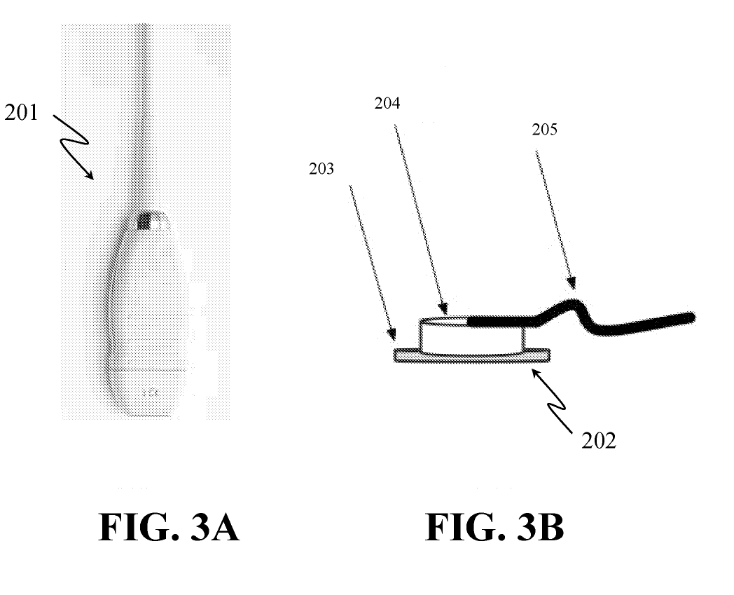

Another embodiment of the present invention is a system and related method for continuous cardiac monitoring. Such a monitoring system would find widespread use in intensive care units and other environments where careful monitoring of cardiac function is essential for patient care. This could display real-time echo images 403 on the screen and/or continuously report cardiac size and function. The monitoring version of the invention preferably does not use the full 12 lead ECG, but would rather uses a three lead or five lead configuration with a specialized low profile ultrasound transducer 202. An exterior view of a 2D array is shown in FIG. 3(A). An exterior view of a low-profile transducer is shown in FIG. 3(B). Other system components are similar to those shown in FIG. 1. Ultrasound coupling gel is generally required to ensure an acoustic match from the transducer through the coupling gel to the human tissue (which is primarily composed of water and has broadly similar acoustic properties). Conventional gel is water-based and is sufficient for conventional ultrasound usage but, for the envisaged application here, the rapid dryout of existing water-based gel is a problem. In one embodiment we propose to replace the water-base gel with an oil-like gel. Ideally the gel will have similar acoustic properties to water. However, if the gel layer is thin, then this requirement is relaxed. An example of a workable oil-based gel is petroleum jelly (Vaseline). In another embodiment the transducer would use a layer of adhesive coupling gel, like that used in ECG tab electrodes such as those manufactured by Nikomed, Cardiosens, and Skintact. Such electrodes dry very slowly and thus a similar gel would work for the ultrasound couplant. In yet another embodiment the ultrasound couplant would include a gel layer and a fluid reservoir so that as the gel dries new fluid from the reservoir replenishes it.

One potential issue with the proposed transducer design and technician workflow is the intrinsic challenge of placing an ultrasound probe properly with limited image guidance and technician training One preferred embodiment of the system mitigates this risk by automatically selecting an active imaging window from a larger transducer array 206. A simple diagram showing such a system appears in FIG. 4. Such a system emits pulses from each individual element to image the area directly under that element. If the region is found to contain a bright reflector very near the transducer, or if no significant echoes are detected from a desired range of interest, then that element is considered “obstructed” and is disabled. A set of “unobstructed” elements is then be used to form images, without the artifacts that would be present if “obstructed” elements had been used for image formation. One of ordinary skill in the art will immediately realize that the above method need not be limited to operation on single array elements, but could be readily applied to groups of elements.

One potential problem with setting the active aperture using the adaptive method described above is the odd active array geometry that would result. Irregular array geometries have the potential to form images with poor contrast and resolution, as such geometries will not intrinsically incorporate proper apodization (needed to reduce side lobes and grating lobes). This limitation may be circumvented by an embodiment of the system that applies apodization design algorithms such as those recently described in the following citations, which are hereby incorporated by reference:

Guenther, D. A., and W. F. Walker, “Broadband Optimal Contrast Resolution Beamforming,” submitted to IEEE Trans. Ultrason. Ferroelec. Freq. Contr., May 2007.

Guenther, D. A., and W. F. Walker, “Optimal Apodization Design for Medical Ultrasound using Constrained Least Squares. Part I: Theory,” IEEE Trans. Ultrason. Ferroelec. Freq. Contr., vol. 54, no. 2, pp. 332-42, February 2007.

Guenther, D. A., and W. F. Walker, “Optimal Apodization Design for Medical Ultrasound using Constrained Least Squares. Part II: Results,” IEEE Trans. Ultrason. Ferroelec. Freq. Contr., vol. 54, no. 2, pp. 343-58, February 2007.

Ranganathan, K. and W. F. Walker, “A Novel Beamformer Design Method for Medical Ultrasound: Part I: Theory,” IEEE Trans. Ultrason. Ferroelec. Freq. Contr., IEEE Trans. Ultrason. Ferroelec. Freq. Contr, vol. 50, no. 1, pp. 15-24, January 2003.

Ranganathan, K. and W. F. Walker, “A Novel Beamformer Design Method for Medical Ultrasound: Part II: Simulation Results,” IEEE Trans. Ultrason. Ferroelec. Freq. Contr., vol. 50, no. 1, pp. 25-39, January 2003.

D. G. Guenther and W. F. Walker, “Optimal Contrast Resolution Beamforming,” presented at the 2007 IEEE Ultrasonics Symposium.

D. A. Guenther and W. F. Walker, “Receive Channel FIR Filters for Improved Contrast in Medical Ultrasound,” 2007 SPIE Medical Imaging Symposium, San Diego, USA.

An aspect of the present invention is the combined ECG/Echo diagnostic report 402 generated by the system. One embodiment of such a report is shown in FIG. 5.

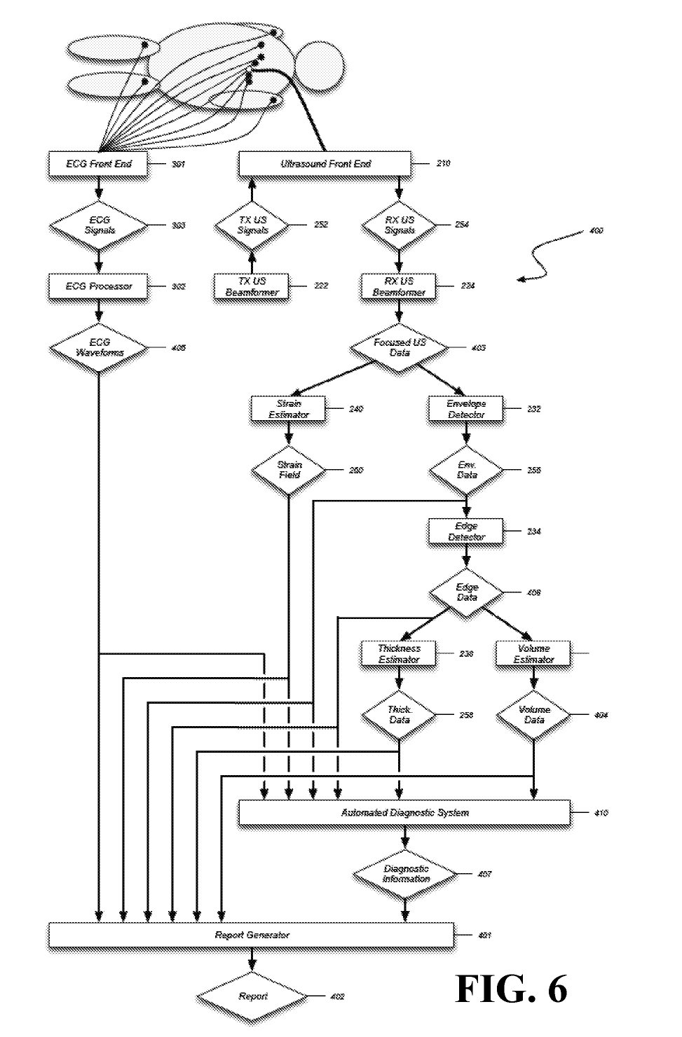

Referring to FIG. 6, an embodiment of the system 400 is shown similar to that in FIG. 1 with the addition of data elements and more detailed exposition of the processing stages. The ECG front-end 301 passes ECG signals 303 to the ECG processor 302 which passes ECG waveforms 406 to the automated diagnostic system 410 and/or the report generator 401. The ultrasound front-end 210 passes transmit ultrasound waveforms 252 generated by the transmit beamformer 222 to the ultrasound transducer (shown as a white circle on the subject). The echoes returned from the transmission are received by the transducer then pass through the ultrasound front-end 210 to yield the received ultrasound signals 254. These signals are then focused by the receive ultrasound beamformer 224 to yield focused ultrasound data 403. Depending upon the specific beamforming algorithm used, the focused ultrasound data may be volumetric in nature, may consist of a set of separate two dimensional images, or may be individual lines of data. In one embodiment the system will use the DSIQ beamforming algorithm and will naturally produce c-scan images.

The focused ultrasound data 403 produced by the ultrasound receive beamformer 224 may be passed into one or more separate data paths. In one path the focused ultrasound data 403 is passed to a strain estimator 240 to yield a strain field 260. In another possible data path the focused ultrasound data 403 is passed to an envelope detector 232 which produces envelope detected ultrasound data 256. Such data contains information about the ultrasound image and the underlying tissue, but lacks phase information. The envelope detected ultrasound data 256 may then be processed by the edge or boundary detector 234 to yield edge or boundary data 408 corresponding to the specific tissues of interest (i.e. myocardium). Edge data 408 may be further processed by the thickness estimator 238 to quantify the thickness 258 of various tissues such as the left ventricular wall, the septum, or the right ventricular wall. The edge data 408 may also or alternatively be passed to a volume estimator 236 to estimate the volume 404 of the ventricles or other tissues of interest. Any or all of the ECG waveforms 406, the strain field 260, the envelope data 256, the boundary data 408, the measured thicknesses 258, and the measured volumes 404 are each passed on to the automated diagnostic system 410. The automated diagnostic system 410 processes these various inputs to determine diagnostic information 407. Diagnostic information 407 along with the various inputs to the automated diagnostic system 410 are passed to the report generator 401. The report generator generates a report 402.

One of ordinary skill in the art will appreciate that the exemplary embodiment described above could be readily modified through the addition of Color Flow Doppler, Tissue Doppler, Integrated Backscatter, Power Mode Doppler, or any of a broad variety of other ultrasound signal and image processing methods.

The present invention diagnostic device shall have an impact on clinical practice. This diagnostic device, system and related method will replace all standard electrocardiographs, since it will no longer be acceptable to infer chamber size and wall thickness by a standard ECG. All uses for the ECG as a tool for diagnosing and following chamber size and hypertrophy will be supplanted by this device (e.g. following the individual with hypertension). This device, system and related method will broaden the indications for screening for heart disease, in populations such as athletes.

Currently, ECG screening is not performed in the US because of the concern of false-positive diagnosis of the ECG being read as left ventricular hypertrophy in an athlete, when in fact it was normal. The presence of the echo function in the device eliminates the false positive diagnosis since it is possible to measure the wall and septal thickness and rule out the diagnosis of hypertrophic cardiomyopathy, which is the dangerous condition that kills athletes.

An aspect of an embodiment of the present invention device shall provide varying degrees of diagnostic echo/Doppler capability. The device shall supplant the ECG for chamber, wall, and septal measurement.

The present invention monitoring device shall have an impact on clinical practice. Such a monitoring device, system, and related method can remain attached to all critically ill patients (e.g. after heart surgery) demonstrating real-time cardiac chamber size and ejection fraction, a measure of cardiac function. Such parameters are vital to the care of ill patients and are currently obtained intermittently by a standard echo; the ability to see a chamber enlarging or function deteriorating early in the course of disease progression or early in the course of the disease would literally save lives. As well, it would be possible to monitor fluid buildup between the heart and pericardium; such buildup can be responsible for cardiac arrest and early diagnosis would prevent catastrophe. It is likely that this monitoring device would become a standard “plug in” to virtually all monitors in Intensive Care Units.

The devices, systems and methods of various embodiments of the invention disclosed herein may utilize aspects disclosed in the following references and patents and which are hereby incorporated by reference herein in their entirety:

1. Multi-Electrode Panel System for Sensing Electrical Activity of the Heart, U.S. Patent Application Pub. No. 2004/0015194 A1, Ransbury, et. al., Jan. 22, 2004.

2. Method of Imaging in Ultrasound Diagnosis and Diagnostic Ultrasound System, U.S. Pat. No. 5,615,680, Akihiro Sano, Apr. 1, 1997.

3. Multi-Electrode Panel System for Sensing Electrical Activity of the Heart, U.S. Pat. No. 6,548,343 B1, Ransbury, et. al., Jun. 24, 2003.

4. Ultrasonic Imaging System and Method for Displaying Tissue Perfusion and Other Parameters Varying with Time, U.S. Pat. No. 6,692,438 B2, Skyba, et. al., Feb. 17, 2004.

5. Systems and Methods for Making Noninvasive Assessments of Cardiac Tissue and Parameters, U.S. Pat. No. 7,022,077 B2, Mourad, et. al., Apr. 4, 2006.

6. Single or Multi-Mode Cardiac Activity Data Collection, Processing and Display Obtained in a Non-Invasive Manner, U.S. Pat. No. 7,043,292 B2, Tarjan, et. al., May 9, 2006.

7. Non-Invasive Method and Device to Monitor Cardiac Parameters, U.S. Pat. No. 7,054,679 B2, Robert Hirsh, May 30, 2006.

8. Single or Multi-Mode Cardiac Activity Collection, Processing and Display Obtained in a Non-Invasive Manner, U.S. Patent Application Pub. No. 2003/0236466 A1, Tarjan, et. al., Dec. 25, 2003.

9. Method and Apparatus for Non-Invasive Ultrasonic Fetal Heart Rate Monitoring, U.S. Patent Application Pub. No. 2005/0251044 A1, Hoctor, et. al., Nov. 10, 2005.

10. Ultrasonic Imaging System and Method for Displaying Tissue Perfusion and Other Parameters Varying with Time, U.S. Pat. No. 6,692,438 B2, Skyba, et al., Feb. 17, 2004.

11. Non-invasive Method and Device to Monitor Cardiac Parameters, U.S. Pat. No. 7,054,679 B2, Robert Hirsh, May 30, 2006.

12. Intuitive Ultrasound Imaging System and Related Method Thereof, U.S. Patent Application Pub. No. 2005/0154303 A1, Walker, et al., Jul. 14, 2005.

13. Multi-Electrode Panel System for Sensing Electrical Activity of the Heart, U.S. Patent Application Pub. No. 2004/015194 A1, Ransbury, et al., Jan. 22, 2004.

An aspect of the present invention yields the electrophysiological measurement functions of an ECG while at the same time performing highly accurate measurements of cardiac chamber volumes, wall and septal thicknesses, and other geometric measures. This yields a more powerful tool for the diagnosis, screening, and monitoring of cardiac conditions. This device will replace all standard electrocardiographs, since it will no longer be acceptable to infer chamber size and wall thickness by a standard ECG. All uses for the ECG as a tool for diagnosing and following chamber size and hypertrophy may be supplanted by this device (e.g. following the individual with hypertension). This device, system and related method will broaden the indications for screening for heart disease, in populations such as athletes. A goal of an embodiment of the present invention is to yield a system that is low in cost and easy to use, to maximize its clinical utility. A further goal of an embodiment of the present invention is a system that is highly portable and therefore appropriate for a broad range of applications.

The low profile transducer and automated volume estimation capabilities of the present invention will enable chronic monitoring of tissue volumes in a variety of applications. Such monitoring would be particularly useful in animal experimentation. In one application the system would be placed over a tumor and tumor volume could be measured serially to assess the impact of various drug regimens or other therapies. In another application the device could be used to serially measure tissue swelling or edema and assess the efficacy of various treatments.

Some exemplary and non-limiting products and services that which the various embodiments of the present invention may be implemented include, but not limited thereto, the following: medical diagnosis, medical monitoring, and screening for disease.

Some exemplary and non-limiting advantages associated with various embodiments of the present invention may include, but not limited thereto, the following: low cost, easy to use, portable, little user dependence, and fast results.

Practice of the invention will be still more fully understood from the following examples, which are presented herein for illustration only and should not be construed as limiting the invention in any way.

### EXAMPLE NO. 1

While the ECG is a simple test that is used to diagnose cardiac hypertrophy and enlargement (ventricles and atria) it is frequently wrong. A way to assess cardiac hypertrophy and enlargement is with an echocardiogram. However, echo requires a trained technician and is expensive.

An attribute of an embodiment of present invention is that the ECG should never again be used to diagnose cardiac hypertrophy and enlargement. It may be used for rate, rhythm, conduction disturbance, infarction, etc., but it should never be called upon to assess cardiac hypertrophy and enlargement.

An approach of an embodiment of the present invention provides an echo transducer and software that automatically measures the size of the heart. A transducer (or small transducer array) would be placed on the chest and a 3-D echo taken, allowing for automated positioning of the image and automated measurement. A technician would not be required. This transducer would be lightweight and possibly disposable. It would be placed at the same time at the ECG leads and a combined ECG/Echo report would be made. The cardiac hypertrophy and enlargement diagnosis (as well as ejection fraction, a measure of cardiac function) would be based upon the echo, and the rest on the ECG.

A significance of an embodiment of this device and method is that it would, but not limited thereto, set the new standard for “electrocardiographs” and potentially, every machine would be converted. Additionally, this technology would be applicable to real-time monitoring in ICU's (continuous ejection fraction and cardiac size), and potential ambulatory monitoring of cardiac hypertrophy and enlargement as well as function.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims, including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

1. A system for obtaining volumetric ultrasound data from a subject, comprising:
an ultrasound transducer assembly including (i) a transmit transducer that transmits ultrasound waves, (ii) a two-dimensional receive transducer array comprising a plurality of elements that receives ultrasound echoes and converts the ultrasound echoes into electrical signals, (iii) receive circuitry for generating a plurality of abbreviated data records for each of the plurality of transducers, wherein each of the abbreviated data records comprises echo data about a desired depth;
an ultrasound beamformer connected via a communication channel to said receive circuitry adapted to create volumetric ultrasound data comprising a plurality of c-mode ultrasound images generated from the abbreviated data records corresponding to a plurality of target depths;
at least one of a real-time display, storage subsystem, or communications channel for outputting or storing said volumetric ultrasound data.

2. The system of claim 1 wherein the receive circuitry for generating a plurality of abbreviated data records comprises at least one of (i) an analog-to-digital converter operating in a burst mode to acquire echo data samples from only the desired target depth and (ii) an analog-to-digital converter operating in direct sampled IQ mode.

3. The system of claim 1 further comprising:
a subsystem for generating tissue boundary data from said volumetric ultrasound data; and,
a subsystem for estimating volume data using said boundary data.

4. The system of claim 1 further comprising:
a subsystem for generating tissue boundary data from said plurality of ultrasound images; and,
a subsystem for estimating thickness data using said boundary data.

5. The system of claim 3 or 4, wherein the tissue boundary is one or more of the inner or outer boundaries of the myocardium.

6. The system of claim 1 further comprising:
a subsystem for estimating tissue strain within and across the plurality of ultrasound images.

7. The system of claim 1 further comprising
a diagnostic electrocardiogram system for acquiring data with a known time relationship to the plurality of ultrasound images.

8. The system of claim 7 wherein the diagnostic electrocardiogram system is either a 12 lead or a 7 lead electrocardiogram.

9. The system of claim 7, further comprising:
a subsystem for generating tissue boundary data from said volumetric ultrasound data,
a subsystem for estimating volume data using said boundary data, and
a real-time display, storage subsystem, or communications channel which has been modified for outputting or storing said volume data in addition to said ultrasound data or result from processing said ultrasound data, and said electrocardiogram data.

10. The system of claim 3 or 9, wherein the volumetric ultrasound data correspond to one or more of a myocardial muscle volume, a left ventricular chamber volume, a right ventricular chamber volume, a left atrial chamber volume, or a right atrial chamber volume.

11. The system of claim 7, wherein said real-time display, storage subsystem, or communications channel is adapted to output or store said ECG signals in addition to said ultrasound data and/or data obtained by processing said ultrasound data.

12. The system of claim 7, further comprising at least one of an additional real-time display, an additional storage subsystem, or an additional communications channel for outputting or storing said ECG signals.

13. The system of claim 1 wherein all of the plurality of elements in the receive transducer array are used as the transmit transducer to emit a planar transmit ultrasound wave.

14. The system of claim 1 further comprising an oil-based transducer couplant.

15. The system of claim 3 or 9, wherein the subsystem for estimating volume data comprises:
a subsystem for determining the area of the region defined by said tissue boundary data;
a subsystem for determining a slice thickness for each of the plurality of ultrasound images; and,
a processor adapted to multiply said area by said slice thickness to obtain a slice volume for each of the plurality of ultrasound images, and to sum said slice volumes to obtain a total volume.

16. The system of claim 9, further comprising a processor adapted to use said volumetric ultrasound data or said ECG signals to generate diagnostic information.

17. The system of claim 7 wherein the transducer assembly incorporates at least one ECG electrode.

18. The system of claim 1 wherein the ultrasound transducer assembly has a subject contact area having at least one linear dimension greater than the thickness of said transducer assembly.

19. The transducer assembly of claim 1 further comprising a flexible cable for connecting the transducer assembly to at least one of the beamformer, display, or power source.

20. The transducer assembly of claim 19 wherein the cable exits the transducer assembly in a plane substantially parallel to the active transducer face.

21. The system of claim 1 further comprising an adhesive transducer couplant to acoustically couple the transducer assembly to a subject.

22. The system of claim 1 further comprising a gel-based transducer couplant connected to a fluid reservoir.

23. The system of claim 1 further comprising a diagnostic report generator for generating diagnostic reports including at least diagnostic ECG waveforms or information extracted from ECG waveforms and ultrasound image data or information extracted from such image data.

24. The system of claim 23 wherein the information extracted from said ultrasound data consists of estimates of a volume of any chambers of a heart.

25. The system of claim 23 wherein the information extracted from said ultrasound data consists of estimates of the thickness of any of the myocardium or the septum of the heart.

26. The system of claim 1 wherein the ultrasound beamformer does not use some or all of the elements that are determined to be obstructed.

27. The system of claim 1 wherein one or more of the plurality of elements in the receive transducer array are used as the transmit transducer.

28. (canceled)

29. (canceled)
